(19) United States
(12) Patent Application Publication
(10) Pub. No.: US 2013/0284609 A1
(43) Pub. Date: Oct. 31, 2013

(54) ENZYMATIC ELECTROCHEMICAL-BASED SENSORS WITH NAD POLYMERIC COENZYME

(71) Applicants: Zuifang LIU, Inverness (GB); Steven Setford, Inverness (GB); Marco Cardosi, Inverness (GB)

(72) Inventors: Zuifang LIU, Inverness (GB); Steven Setford, Inverness (GB); Marco Cardosi, Inverness (GB)

(73) Assignee: Lifescan Scotland Limited

(21) Appl. No.: 13/460,236

(22) Filed: Apr. 30, 2012

(51) Int. Cl. G01N 27/327; G01N 33/50

(52) U.S. Cl. 205/777.5; 204/403.14

(57) ABSTRACT

A nicotinamide adenine dinucleotide (NAD) polymeric coenzyme for use in enzymatic electrochemical-based sensors includes NAD moieties covalently bound as pendent groups to a polymer backbone. An enzymatic electrochemical-based biosensor includes nicotinamide adenine dinucleotide (NAD) polymeric coenzyme, a polymeric electron transfer agent (e.g., polymeric ferrocene) at least one working electrode, and at least one reference electrode.

### BACKGROUND OF THE INVENTION

1. Field of the Invention

The present invention relates, in general, to medical devices and, in particular, to enzymatic electrochemical-based sensors.

2. Description of Related Art

The determination (e.g., detection and/or concentration measurement) of an analyte in a fluid sample is of particular interest in the medical field. For example, it can be desirable to determine glucose, ketone bodies, lactate, cholesterol, lipoproteins, triglycerides, acetaminophen and/or HbA1c concentrations in a sample of a bodily fluid such as urine, blood, plasma or interstitial fluid. Such determinations can be achieved using sensors, based on, for example, visual, photometric or electrochemical techniques. Conventional electrochemical-based analytical test strips are described in, for example, U.S. Pat. Nos. 5,708,247, and 6,284,125, each of which is hereby incorporated in full by reference.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1 is a simplified chemical sequence for the synthesis of functionalized nicotinamide adenine dinucleotide (NAD);

FIG. 2 is a simplified chemical sequence for the synthesis of an NAD monomer;

FIG. 3 is a simplified chemical sequence for the synthesis of a NAD polymeric coenzyme employed in embodiments of the present invention;

FIG. 4 is a graph depicting the current response to β-hydroxybutyric acid of an enzymatic electrochemical-based biosensor that includes a NAD polymeric coenzyme employed in embodiments of the present invention;

FIGS. 5A, 5B, and 5C are simplified cross-sectional end, perspective, and exploded perspective views of an enzymatic electrochemical-based sensor (i.e., an electrochemical based analytical test strip) according to an embodiment of the present invention; and

FIG. 6 is a flow diagram depicting stages in a method for determining an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, nicotinamide adenine dinucleotide (NAD) polymeric coenzymes that are employed in enzymatic electrochemical-based sensors according to embodiments of the present invention include NAD moieties covalently bound as pendent groups to a polymer backbone (e.g. polyacrylamide). Such NAD polymeric coenzymes are beneficial in that they can be readily incorporated as a redox coenzyme into enzymatic electrochemical-based biosensors (such as analytical test strips) according to embodiments of the present invention by employing, for example, techniques to immobilize the polymeric NAD coenzyme and a polymeric electron transfer agent within an analyte detection matrix. Such enzymatic electrochemical-based biosensors include continuous biosensors and beneficially combine NAD polymeric coenzymes with polymeric electron transfer agents (see, for example, U.S. Patent Publication 2006/0069211, which is hereby incorporated in full be reference).

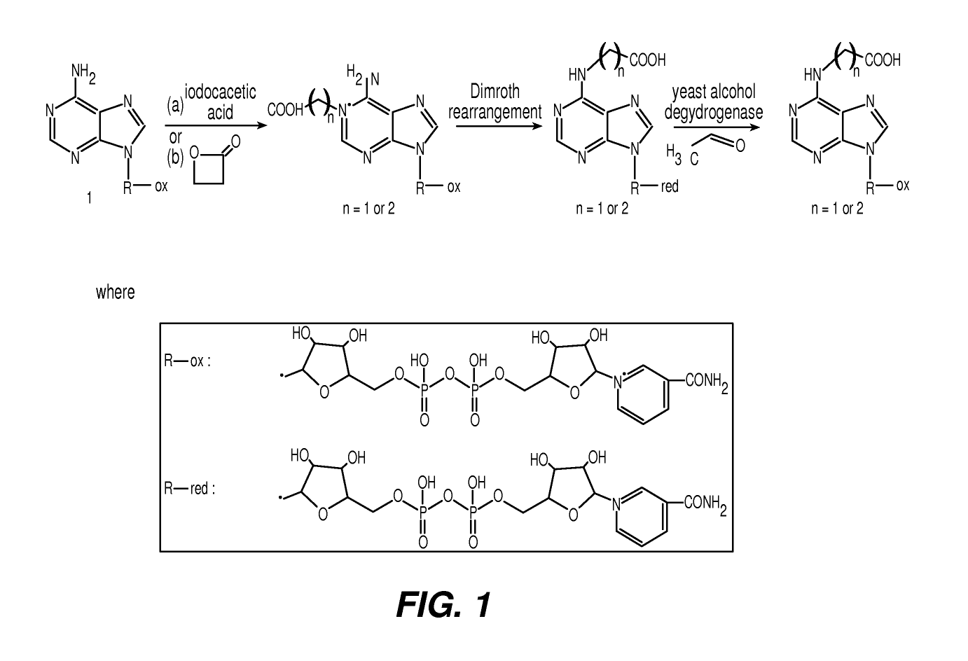

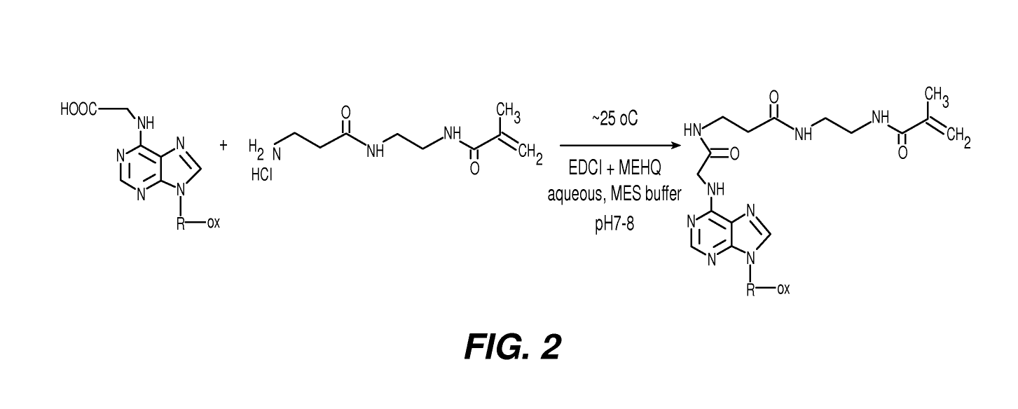

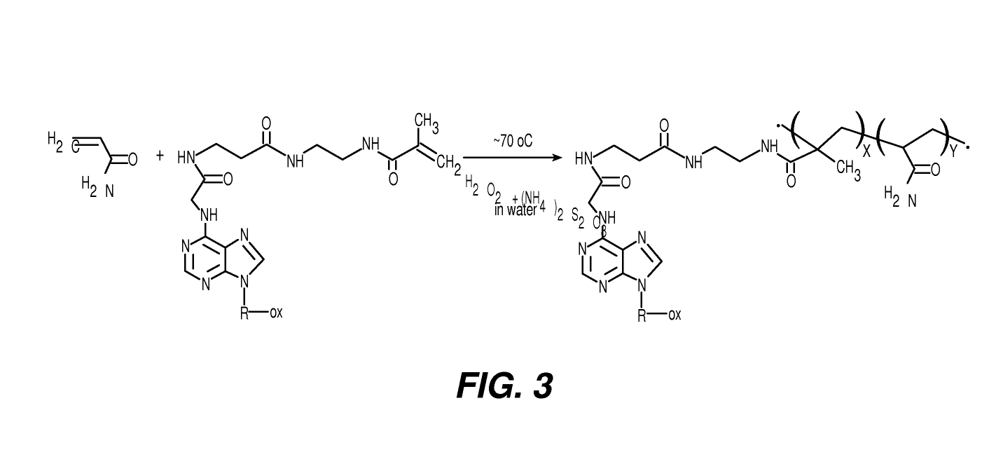

FIG. 1 is a simplified chemical sequence for the synthesis of functionalized nicotinamide adenine dinucleotide (NAD), where “n” (as employed in FIG. 1)=1 or 2. FIG. 2 is a simplified chemical sequence for the synthesis of an NAD monomer where “n” (as employed in FIG. 1)=1. FIG. 3 is a simplified chemical sequence for the synthesis of a NAD polymeric coenzyme that can be employed in electrochemical-based biosensor embodiments of the present invention. As employed in FIG. 3, “X” represents the number of NAD monomer repeating units, while “Y” represents the number of repeating acrylamide units. The number of NAD monomer repeating units (i.e. “X”) can be any suitable number.

Once appraised of the present disclosure, one skilled in the art will recognize that although FIGS. 1 through 3 depict a particularly beneficial NAD polymeric coenzyme that includes acrylamide co-monomers in the backbone, other suitable monomer(s) can also be employed. Such suitable monomers can include but are not limited to, for example, hydroxyethyl methacrylate, vinylpyrrolidon, (3-(methacryloylamino) propyl) trimethyl ammonium chloride, (2-methacryloyloxy) ethyl) trimethyl ammonium chloride, sodium-4-styrene sulfonate, acrylic acid, N,N′-diethylacrylimide, and N,N′-dimethylacrylamide.

The synthesis depicted in those figures are described below with reference to the figures. The synthesis of N⁶-carboxymethyl-NAD⁺ was accomplished using the following 3 step process:

### Step 1: Synthesis of N¹-carboxymethyl-NAD⁺ (the second structure of FIG. 1 with n=1)

To a 5 ml Biotage microwave reaction tube, added NAD⁺ (1.0 g, 1.51 mmol) pre-dissolved in 0.1M pH 7.0 sodium phosphate buffer (3.5 ml) and iodoacetic acid (1.5 g, 8.06 mmol, 5.34 eq). The pH was adjusted to 7.0 by using 5.0M NaOH aqueous solution. The reaction vessel was sealed and the mixture was heated to 50° C. for 10 minutes using microwave irradiation.

The resultant pink solution (c.a. 5 ml) was acidified to pH3.0 using 5M HCl aqueous solution before being poured into a pre-cooled (−5° C.) mixture of acetone/IMS (1:1) (25 ml). The resulting precipitate was filtered, washed first with IMS (5 ml), then dry diethyl ether (15 ml) before air drying under dry nitrogen for 10 minutes. Further drying overnight in a desiccator over fused CaCl₂ afforded N1-carboxymethyl-NAD⁺ as a pink amorphous solid (1.62 g) (crude).

### Step 2: Synthesis of N⁶-carboxymethyl-NADH (the third structure of FIG. 1 with n=1)

The above prepared crude N¹-carboxymethyl-NAD⁺ (9.1 g, c.a. 10.57 mmol) was dissolved in 1.3% w/v NaHCO₃ in aqueous solution (450 ml) and the solution deoxygenated by sparging with nitrogen for 10 minutes. Sodium dithionite (3.5 g, 20.1 mmol) added in one portion and the mixture stirred at ambient temperature to effect reduction of the nicotinamide moiety (i.e. conversion of oxidized state NAD+ to reduced state NADH). After 1.0 hour, the solution color had changed from pink to yellow. The solution was then sparged with air for 10 minutes to destroy any excess dithionite and the pH brought to 11.0 by using 5M NaOH aqueous solution. The mixture was heated at 70° C. for 90 minutes, to promote Dimroth rearrangement to N⁶-carboxymethyl-NADH, before cooling to 25° C. Thin-layer chromatography (silica gel, isobutryic acid/water/32% NH₄OH (aq), 66/33/1.5 by volume) showed no evidence for the presence of N¹-carboxymethyl-NADH at this stage.

### Step 3: Oxidation of N⁶-carboxymethyl-NADH to N⁶-carboxymethyl-NAD⁺ (the fourth structure of FIG. 1 with n=1)

The reaction mixture containing N⁶-carboxymethyl-NADH was treated with 3M TRIS buffer (pH7.0) (17.5 ml) and the pH adjusted to 7.5 using 5M HCl aqueous solution (c.a. 4.9 ml). Acetaldehyde (3.5 ml, 62.6 mmol) was added, immediately followed by yeast alcohol dehydrogenase (from saccharomyces cerevisiae) (˜300 U/mg) (10.5 mg, c.a. 3150 U of enzyme) before allowing to stir at ambient temperature to deoxidize the nicotinamide moiety (i.e. conversion of NADH to NAD⁺). After 18 hours, the reaction mixture (c.a. 485 ml) was concentrated in vacuo (30° C./10-15 bar) to approximately ⅓ volume and poured into a pre-cooled (−5° C.) mixture of acetone/IMS (1:1) (1800 ml). The fine slurry was left to age for 18 hours at 3° C. The resulting precipitate was collected by centrifugation and washed on a glass sinter with IMS (40 ml) then dry diethyl ether (120 ml) before air-drying under dry nitrogen for 10 minutes. Further drying overnight in a desiccator over fused CaCl₂ afforded N⁶-carboxymethyl-NAD⁺ as a tan colored hygroscopic solid (3.99 g) (crude).

The crude N⁶-carboxymethyl-NAD⁺ (1.0 g) was taken up in water (20 ml) and passed through a Sephadex G10 gel filtration column (2×10 cm, 20 ml). All eluted fractions containing UV active material were combined (60 ml total volume) and added to a column of Dowex 1-X2 ion exchange resin (Cl⁻; 4×50 cm, 200 ml) which had been pre-equilibrated with water. A linear gradient of 0-50 mM LiCl (buffered to pH 3.0), at 10 ml/min over 65 min, was applied using “Presearch Combiflash Companion” chromatography equipment. The fractions eluted between 25-35 mM were combined (c.a. 100 ml), neutralized to pH 7.0 with 5M LiOH and evaporated to approximately ⅓ volume and poured into a pre-cooled (−5° C.) mixture of acetone/IMS (1:1) (300 ml). The fine slurry was left to age for 18 hours at 3° C. The resulting precipitate was collected by centrifugation and washed on a glass sinter with IMS (30 ml) then dry diethyl ether (50 ml) before air-drying under dry nitrogen for 10 minutes. Further drying overnight in a desiccator over fused CaCl₂ afforded purified N⁶-carboxymethyl-NAD⁺ as a cream colored hygroscopic solid (0.307 g). Extrapolated total yield=1.225 g, 14%.

NAD monomer was prepared as follows and as depicted in FIG. 2. A solution of 1-ethyl-3-(3-dimethylaminopropyl) carbodiimide (EDCI) (0.2 g, 1.29 mmol, 1.0 mol eq) in water (20 ml), at ambient temperature, was adjusted to pH 7.2, using 2.0M HCl aqueous solution. N⁶-carboxymethyl-NAD⁺ (0.45 g, 0.622 mmol, 0.5 mol eq) and hydroquinone (0.017 g, 0.154 mmol, 0.125 mol eq) were added in sequentially, single portions, and the pH adjusted to pH 7.2 using 2.0M HCl aqueous solution. Reagent N⁶—[N-(2-(N-(2-methacrylamidoethyl) carbamoylmethyl)-NAD⁺ was added in a single portion, at ambient temperature, and the pH adjusted to pH 7.2, using 2.0M HCl aqueous solution. The mixture was stirred at ambient temperature, in the dark, for 24 hours.

The pH was monitored and maintained at between pH7.0 and pH7.5. The reaction mixture was then diluted with distilled water (30 ml) and passed through a pre-prepared bed of Sephadex gel (15 g equilibrated with 150 ml distilled water) and eluted with distilled water (80 ml). The eluate (approximately 110 ml) was loaded onto a pre-prepared column (Dowex 1-X2 (Cl⁻ form)) which was set up to run through an automated chromatography system (Presearch Combiflash Companion) and was equilibrated with 5 column volumes of water at 40 ml/minute. The flow rate was reduced to 20 ml/minute prior to loading the crude compound (via the pump).

A linear gradient of 0-50 mM of aqueous lithium chloride (buffered at pH3) over 60 minutes was applied (i.e. binary pump configuration; A: water, B: 50 mM LiCl (pH 3), 100:0 A:B to 0:100 A:B over 60 min). The eluted fractions were analysed by TLC and the desired fractions (eluted between 40-50 mM, 50 ml) concentrated to half volume (25 ml) and added dropwise to a stirred solution of ethanol (375 ml, 15 volumes) at 2° C. The resulting precipitate was aggregated by centrifugation (Genevac EZ2⁺, 3600 rpm, 3 minutes) collected by filtration and washed with cold ethanol (50 ml). The precipitate was dried at ambient temperature under vacuum, in a desiccator over calcium chloride and subsequently stored in the freezer.

The synthesis of copolymer of the NAD monomer and acrylamide (i.e., an NAD polymeric coenzyme according to an embodiment of the present invention) was conducted as follows and depicted in FIG. 3. 0.209 g the above prepared NAD monomer, 0.8 g acrylamide and 9.2 g water were added to a flask which was equipped with a mechanic stirrer, a condenser, nitrogen inlet and outlet. After deoxygenating with nitrogen for 30 minutes at room temperature, the solution was heated to 70° C., then 50 micro-litre 20 vol solution of hydrogen peroxide and 9 mg ammonium persulfate were added to the flask. The polymerization continued for 6 hours under continuous agitation. The crude polymer was purified by dialysis against water for 2 days and then freeze dried.

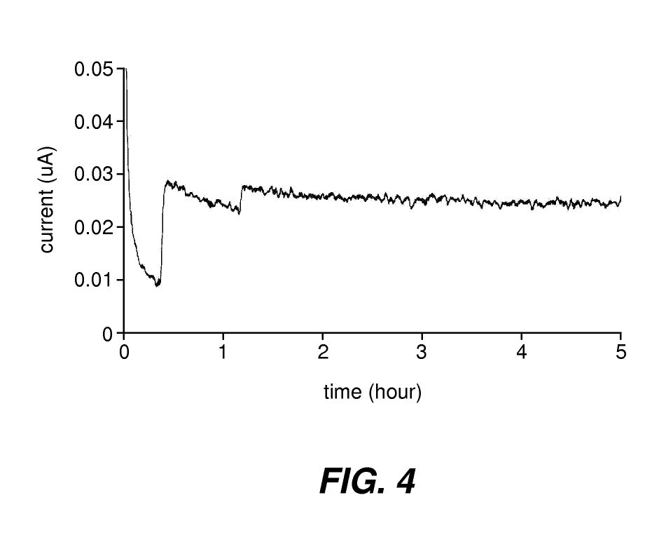

FIG. 4 is a graph depicting the current response of an enzymatic electrochemical-based biosensor according to an embodiment of the present invention that includes both NAD polymeric coenzyme and ferrocene polymeric mediator. The data of FIG. 4 was generated using the following test set-up: (i) a carbon electrode with surface deposited reagent layer. The carbon electrode was fabricated by screen-printing and has an exposure surface dimension of 2.5×2.5 mm defined by an insulation layer. The reagent layer was prepared by air-drying 10 μL (micro liter) solution that contained 2.0 mg/mL of the NAD polymeric coenzyme, 2.0 mg/mL ferrocene polymeric mediator, 1.0 mg/mL β-hydroxybutyrate dehydrogenase, 1.0 mg/mL diaphorase in 0.1M Tris buffer (pH7.4). The reagent coated electrode was soaked in 0.1M Tris buffer (pH7.4) overnight and rinsed with fresh Tris buffer prior to test. (ii) an Ag/AgCl and a platinum wire were used as a reference electrode and counter electrode, respectively.

The measurement of FIG. 4 started with the three electrodes in 10.0 mL of 0.1M Tris buffer (pH 7.4) in a 25 ml beaker. A potential of 0.3V was then applied. To the beaker were added 1.0 mM β-hydroxybutyric acid (HBA, ketone bodies). Magnetic agitation was applied immediately for a short time after HBA addition. A current increase was clearly detected after adding HBA (see FIG. 4) indicating a sensor current response involving polymeric NAD coenzyme and ferrocene polymeric mediator.

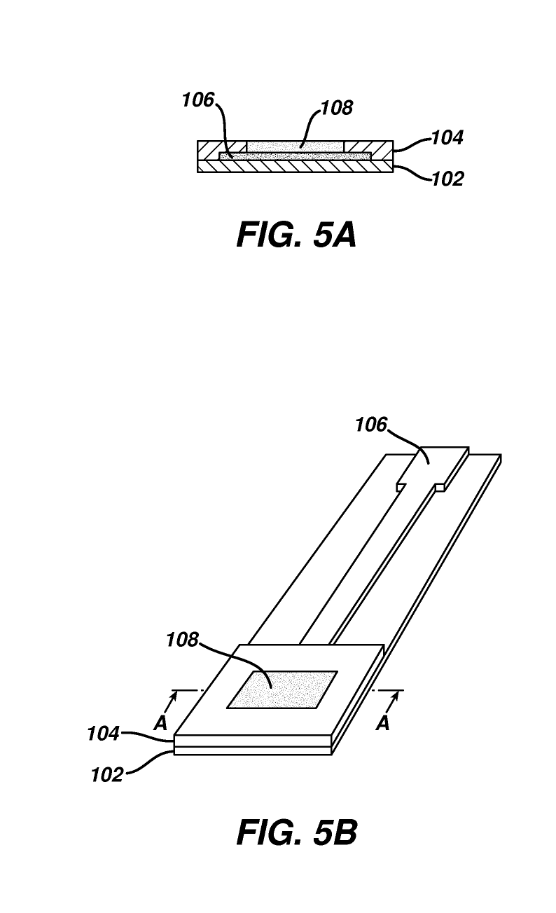

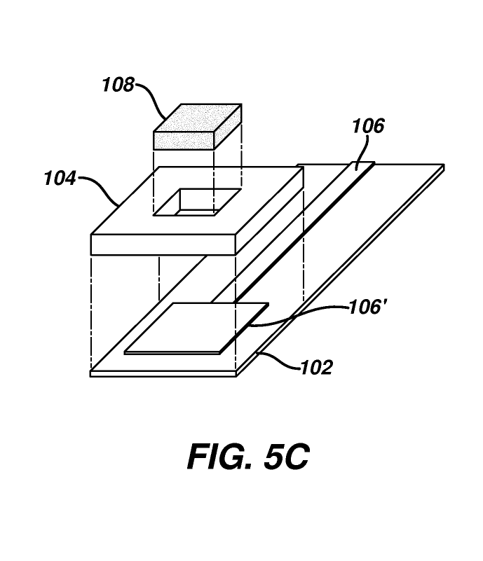

FIGS. 5A 5B, and 5C are simplified cross-sectional end and, perspective and exploded perspective views of an enzymatic electrochemical-based sensor 100 (i.e., an electrochemical based analytical test strip) according to an embodiment of the present invention.

Referring to FIGS. 5A, 5B and 5C, enzymatic electrochemical-based analytical test strip 100 according to an embodiment of the present invention includes an electrically-insulating substrate layer 102, a patterned insulation layer 104, a patterned conductor layer 106 defining at least one working electrode and at least one counter/reference electrode (for clarity not depicted in FIG. 5C as a single component 106′), and an enzymatic reagent layer 108 that includes an NAD polymeric coenzyme as described herein (for example, with respect to FIGS. 1 through 3), a polymeric electron transfer agent (such as polymeric ferrocene) and an enzyme (for example, β-hydroxybutyrate dehydrogenase).

Electrically-insulating substrate layer 102 can be any suitable electrically-insulating substrate known to one skilled in the art including, for example, a nylon substrate, polycarbonate substrate, a polyimide substrate, a polyvinyl chloride substrate, a polyethylene substrate, a polypropylene substrate, a glycolated polyester (PETG) substrate, or a polyester substrate. The electrically-insulating substrate can have any suitable dimensions including, for example, a width dimension of about 5 mm, a length dimension of about 27 mm and a thickness dimension of about 0.5 mm.

Patterned insulation layer 104 can be formed, for example, from a screen printable insulating ink. Such a screen printable insulating ink is commercially available from Ercon of Wareham, Mass. U.S.A. under the name “Insulayer.”

Patterned conductor layer 106 can be formed of any suitable electrically conductive material including, but not limited to, electrically conductive carbon ink materials.

Enzymatic reagent layer 108 can include, in addition to the aforementioned NAD polymeric coenzyme and polymeric electron transfer agent, any suitable enzymatic reagents, with the selection of enzymatic reagents being dependent on the analyte to be determined. For example, if glucose is to be determined in a blood sample, enzymatic reagent layer 108 can include NAD-dependent glucose dehydrogenase along with other components necessary for functional operation. Further details regarding enzymatic reagent layers, and electrochemical-based analytical test strips in general, are in U.S. Pat. No. 6,241,862, the contents of which are hereby fully incorporated by reference.

The polymeric electron transfer agent can be any suitable polymeric electron transfer agent including, for example, a high molecular weight redox polymer comprising a hydrophilic polymer with ionic portions and a plurality of attached redox mediators (for example, ferrocene). The molecular weight of such an ionic hydrophilic high molecular weight polymer can beneficially be, for example, greater than 16 Kg/mol. Such ionic hydrophilic high molecular weight polymers are described in U.S. Patent Publication 2006/0069211, which is hereby incorporated in full be reference.

Electrochemical-based biosensors according to embodiments of the present invention are particularly beneficial in that, for example, the inclusion of both a nicotinamide adenine dinucleotide (NAD) polymeric coenzyme and a polymeric electron transfer agent enables the novel use of dehydrogenase enzymes in a continuous biosensor by, for example, employing them both in an immobilized configuration.

Electrochemical-based analytical test strip 100 can be manufactured, for example, by the sequential aligned formation of patterned insulation conductor layer 106, patterned insulation layer 104 and enzymatic reagent layer 108. Any suitable techniques known to one skilled in the art can be used to accomplish such sequential aligned formation, including, for example, screen printing, photolithography, photogravure, chemical vapour deposition and tape lamination techniques.

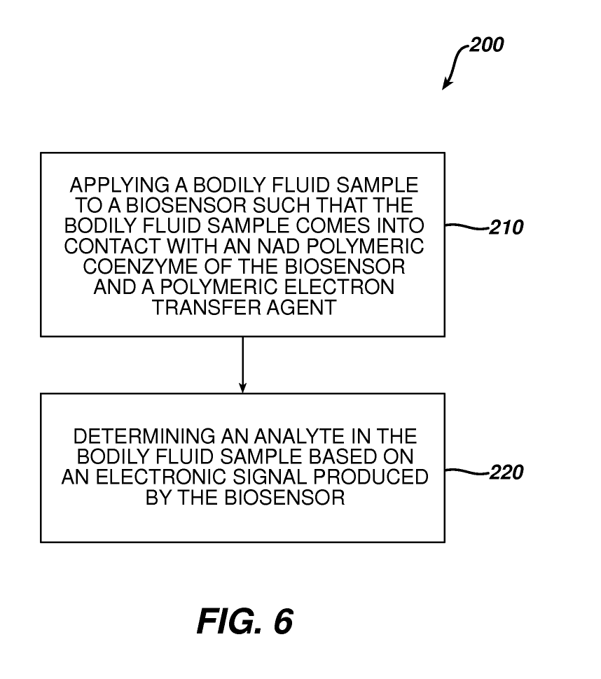

FIG. 6 is a flow diagram depicting stages in a method 200 for determining an analyte (such as glucose or ketone) in a bodily fluid sample (e.g., a whole blood or interstitial fluid sample) according to an embodiment of the present invention. At step 210, method 200 includes applying a bodily fluid sample to an enzymatic electrochemical-based biosensor (e.g., an enzymatic electrochemical-based analytical test strip) such that the bodily fluid sample comes into contact with an NAD polymeric coenzyme that includes NAD moieties covalently bound as pendant groups to a polymer backbone and into contact with a polymeric electron transfer agent (e.g., polymeric ferrocene).

Method 200 further includes determining the analyte in the bodily fluid sample based on an electronic signal produced by the enzymatic electrochemical-based biosensor (see step 220 of FIG. 11).

Once apprised of the present disclosure, one skilled in the art will recognize that method 600 can be readily modified to incorporate any of the techniques, benefits and characteristics of enzymatic electrochemical-based biosensors according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that compositions, devices and methods within the scope of these claims and their equivalents be covered thereby.

What is claimed is:

1. An enzymatic electrochemical-based biosensor comprising:
a nicotinamide adenine dinucleotide (NAD) polymeric coenzyme including NAD moieties covalently bound as pendent groups to a polymer backbone;
a polymeric electron transfer agent;
at least one working electrode; and
at least one reference electrode.

2. The enzymatic electrochemical-based biosensor of claim 1 wherein the NAD polymeric coenzyme and the polymeric electron transfer agent are both in an immobilized configuration.

3. The enzymatic electrochemical-based biosensor of claim 1 wherein the polymer backbone includes predetermined monomers.

4. The enzymatic electrochemical-based biosensor of claim 3 wherein the predetermined monomers are acrylamide monomers.

5. The enzymatic electrochemical-based biosensor of claim 3 wherein the predetermined monomer is selected from the monomer group consisting of hydroxyethyl methacrylate, vinylpyrrolidon, (3-(methacryloylamino)propyl)trimethyl ammonium chloride, (2-methacryloyloxy)ethyl)trimethyl ammonium chloride, sodium-4-styrene sulfonate, acrylic acid, N,N′-diethylacrylimide, and N,N′-dimethylacrylamide.

6. The enzymatic electrochemical-based biosensor of claim 1 wherein the NAD polymeric coenzyme has the following chemical structure: 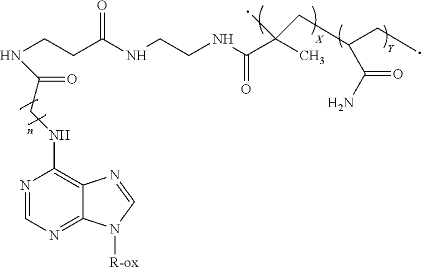
where:
and R in an oxidized form (R-ox) has the following chemical structure: 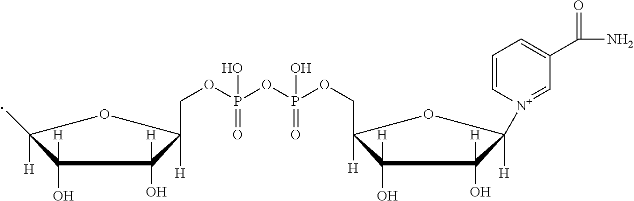
and R in a reduced form (R-red) has the following chemical structure:

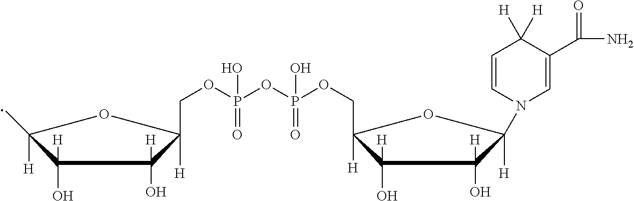

7. The enzymatic electrochemical-based biosensor of claim 6 wherein n equals 1.

8. The enzymatic electrochemical-based biosensor of claim 6 wherein n equals 2

9. The enzymatic electrochemical-based biosensor of claim 1 wherein the NAD polymeric coenzyme has a MW in the range of 1,000 kg/mol to 1,000,000 kg/mol.

10. The enzymatic electrochemical-based biosensor of claim 1 wherein the NAD polymeric coenzyme is in a reduced form.

11. The enzymatic electrochemical-based biosensor of claim 1 wherein the NAD polymeric coenzyme is in an oxidized form.

12. The enzymatic electrochemical-based biosensor of claim 1 wherein the NAD polymeric coenzyme is structured as a redox coenzyme.

13. The enzymatic electrochemical-based biosensor of claim 1 wherein the polymeric electron transfer agent is polymeric ferrocene.

14. The enzymatic electrochemical-based biosensor of claim 1 wherein the polymeric electron transfer agent is a high molecular weight redox polymer comprising:
a hydrophilic polymer that includes ionic portions; and
a plurality of attached redox mediators,
wherein the molecular weight of the ionic hydrophilic high molecular weight polymer is greater than 16 Kg/mol.

15. The enzymatic electrochemical-based biosensor of claim 14 wherein the redox mediator is ferrocene.

16. A method for determining an analyte in a bodily fluid sample, the method comprising:
applying a bodily fluid sample to an enzymatic electrochemical-based biosensor such that the bodily fluid sample comes into contact with a nicotinamide adenine dinucleotide (NAD) polymeric coenzyme that includes NAD moieties covalently bound as pendent groups to a polymer backbone and into contact with a polymeric electron transfer agent; and
determining the analyte based on an electronic signal produced by the biosensor.

17. The method of claim 16 wherein the enzymatic electrochemical-based biosensor is an enzymatic electrochemical-based analytical test strip.

18. The method of claim 16 wherein the analyte is β-hydroxybutyrate.

19. The method of claim 16 wherein the polymer backbone includes predetermined monomers.

20. The method of claim 19 wherein the predetermined monomers are acrylamide monomers.

21. The method of claim 19 wherein the predetermined monomer is selected from the monomer group consisting of hydroxyethyl methacrylate, vinylpyrrolidon, (3-(methacryloylamino) propyl)trimethyl ammonium chloride, (2-methacryloyloxy)ethyl)trimethyl ammonium chloride, sodium-4-styrene sulfonate, acrylic acid, N,N′-diethylacrylimide, and N,N′-dimethylacrylamide.

22. The method of claim 16 wherein the NAD polymeric coenzyme has the following chemical structure: 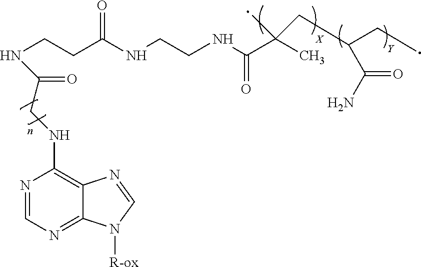
where:
and R in an oxidized form (R-ox) has the following chemical structure: 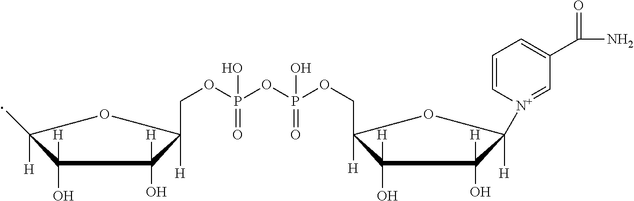
and R in a reduced form (R-red) has the following chemical structure:

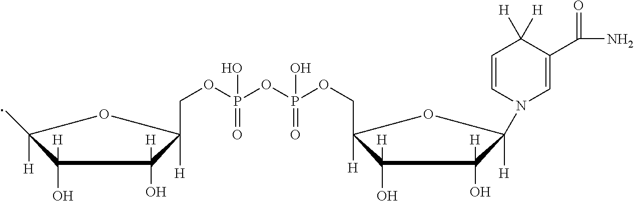

23. The method of claim 22 wherein n equals 1.

24. The method of claim 22 wherein n equals 2.

25. The method of claim 16 wherein the NAD polymeric coenzyme has a MW in the range of 1,000 kg/mol to 1,000,000 kg/mol.

26. The method of claim 16 wherein the NAD polymeric coenzyme is in a reduced form.

27. The method of claim 16 wherein the NAD polymeric coenzyme is in an oxidized form.

28. The method of claim 16 wherein the NAD polymeric coenzyme is structured as a redox coenzyme.

29. The method of claim 16 wherein the polymeric electron transfer agent is polymeric ferrocene.

30. The method of claim 16 wherein the polymeric electron transfer agent is a high molecular weight redox polymer comprising:
a hydrophilic polymer that includes ionic portions; and
a plurality of attached redox mediators,
wherein the molecular weight of the ionic hydrophilic high molecular weight polymer is greater than 16 Kg/mol.

31. The method of claim 30 wherein the redox mediator is ferrocene.

32. The method of claim 16 wherein the NAD polymeric coenzyme and the polymeric electron transfer agent are both in an immobilized configuration.
